SEQUENCE LISTING (4 sequences)

SEQ ID NO: 1 - DNA, 20 bp, artificial sequence
  primer
atggtgagcagagtgcccta

SEQ ID NO: 2 - DNA, 21 bp, artificial sequence
  primer
gtggtgctggaagcctctcct

SEQ ID NO: 3 - DNA, 21 bp, artificial sequence
  primer
ggtgaccaagttcatgctgtg

SEQ ID NO: 4 - DNA, 20 bp, artificial sequence
  primer
gtgtccttgatccacttccg

(19) United States
(12) Patent Application Publication
(10) Pub. No.: US 2011/0178171 A1
(43) Pub. Date: Jul. 21, 2011

(54) COMPOUNDS FOR CANCER THERAPY

(75) Inventors: Yi-Lin CHEN, I-Lan (TW); Horng-Jyh Harn, Taichung City (TW); Shinn-Zong Lin, Taichung City (TW); Tzyy-Wen Chiou, Hualien (TW); Cheng-Tung Lin, Taichung City (TW)

(73) Assignee: China Medical University, Taichung City (TW)

(21) Appl. No.: 13/008,597

(22) Filed: Jan. 18, 2011

Related U.S. Application Data
(60) Provisional application No. 61/296,109, filed on Jan. 19, 2010.

(30) Foreign Application Priority Data
Jul. 15, 2010 (TW) 099123252
Sep. 10, 2010 (TW) 099130636

(51) Int. Cl. A61K 31/365; A61P 35/00

(52) U.S. Cl. 514/464

(57) ABSTRACT

A method of inhibiting the cellular proliferation of at least one selected from the group consisting of androgen dependent prostate cancer cells, androgen independent prostate cancer cells, oral cancer cells, liver cancer cells (hepatoma), and gastric cancer cells in a subject is provided, wherein the method comprises administrating to the subject an effective amount of an active component selected from the group consisting of Z form isochaihulactone (Z-K8) of the following formula (I), E form isochaihulactone (E-K8) of the following formula (II), a pharmaceutically acceptable salt of Z-K8 or E-K8, a pharmaceutically acceptable ester of Z-K8 or E-K8, and combinations thereof:

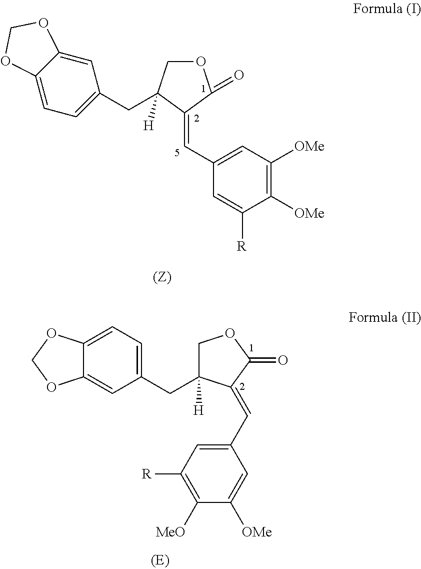

and R is H, alkoxy, or aryl. Also provided is a method for manufacturing Z-K8 and E-K8.

### RELATED APPLICATIONS

The present invention claims priority to Taiwan Patent Application Nos. 099123252, filed on Jul. 15, 2010 and 099130636, filed on Sep. 10, 2010. This application takes priority from Provisional Patent Application 61/296,109 filed on Jan. 19, 2010.

### FIELD

The present invention relates to the use of isochaihulactone in the inhibition of at least one selected from the group consisting of androgen dependent prostate cancer cells, androgen independent prostate cancer cells, oral cancer cells, gastric cancer cells, and liver cancer cells (hepatoma) in a subject.

### BACKGROUND

Cancer is a class of diseases characterized by uncontrolled cellular proliferation, invasion, and metastasis. A major health problem, it caused about 7.6 million human deaths in 2007. Prostate cancer is the most common malignancy diagnosed in American males.

The detection and treatment of the disease is highly successful when tumors are confined to the prostate with their growth and survival strongly androgen-dependent (AD). In this early stage, androgen suppression (or ablation) adopting hormone therapy, for example, using luteinizing hormone-releasing hormone agonists (LHRH agonists) or anti-androgens (e.g., flutamide or cyproterone acetate), is effective. However, if left undetected and untreated, many prostate tumors will become androgen-independent (AI) and/or metastatic, and these advanced-stage tumors are associated with high mortality rates due to ineffectiveness of hormone therapy.

On the other hand, chemotherapy is also ineffective to prostate cancer. Because most agents for chemotherapy aim to kill tumors with high proliferation rate, but prostate cancer cells grow slowly, the agents are inactive to prostate tumors.

Considering the above limitation of hormone therapy and chemotherapy, there is a need for therapies for cancer, such as prostate cancer. The inventors of the present invention found that isochaihulactone may inhibit prostate cancer cells, including androgen dependent and androgen independent prostate cancer cells, and thus this compound is useful in the treatment of prostate cancer. Besides, isochaihulactone is also useful in the treatment of oral cancer, gastric cancer, and liver cancer (hepatoma).

### SUMMARY

This invention relates to isochaihulactone (K8) compounds and methods for treating cellular proliferative disorders, e.g., cancers. Shown below are the structures of Z form and E-form of isochaihulactone (Z-K8 and E-K8), respectively:

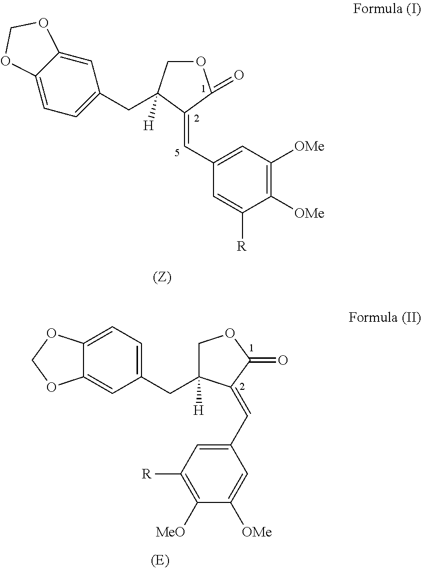

, wherein R is H, an alkoxy, or an aryl.

In one aspect, the invention features a method for manufacturing racemic mixtures of isochaihulactone (K8), i.e., the Z form or E-form of isochaihulactone. The method comprises:

(a) providing a first solution, which is a dichloromethane solution containing a first compound of the following formula (III) and anhydrous triethylamine,

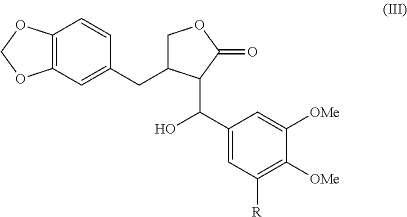

wherein, R is H, methoxy, or an aryl;

(b) providing a second solution, which is an anhydrous triethylamine solution containing methanesulfonyl chloride;

(c) mixing the first solution and the second solution under an inert gas atmosphere to provide a first reacting mixture;

(d) adding the first reacting mixture into a hydrochloric acid to provide a second reacting mixture, and extracting the second reacting mixture with dichloromethane or ethyl estate and collecting organic phase to provide a first extract;

(e) drying the first extract to provide a residue; and

(f) dissolving the residue and a second compound into acetonitrile to carry out an reaction to obtain the racemic mixture, wherein the second compound is selected from a group consisting of 1,8-diazabicyclo[5.4.0]undec-7-ene (DB U), 1,4-diazabicyclo[2.2.2]octane (DABCO) and a combination thereof.

In another aspect, this invention features a composition containing the Z form of isochaihulactone, or the E form of isochaihulactone. The composition can be used in treating a cellular proliferative disorder or regulating the expression of genes involved in cancer.

Thus, another aspect of this invention features a method for treating a cellular proliferative disorder in a subject. The method includes administering to a subject in need thereof an effective amount of the Z form, E form, or both of isochaihulactone. The cellular proliferative disorder can be prostate cancer, oral cancer, gastric cancer, lung cancer, hepatoma, colon cancer, or glioblastoma multiformis. Specifically, this invention provides a method of inhibiting the cellular proliferation of at least one selected from the group consisting of androgen dependent prostate cancer cells, androgen independent prostate cancer cells, oral cancer cells, gastric cancer cells, and liver cancer cells (hepatoma) in a subject, comprising administrating to the subject an effective amount of an active component selected from the group consisting of Z-K8, E-K8, pharmaceutically acceptable salts of Z-K8 or E-K8, pharmaceutically acceptable esters of Z-K8 or E-K8, and combinations thereof.

In yet another aspect, this invention features a method for reducing in a cell or in a subject the expression level of the gene encoding androgen receptor (AR), the EGR-1 gene, or the NAG-1 gene. The method includes contacting the cell with the Z or E form of isochaihulactone, or administering the compound to the subject. The method can further include a step of determining the expression level of one or more of the genes to confirm the inhibition after the contacting or administration step. In a preferred embodiment, the Z form of isochaihulactone is substantially more than the E form in the above-described composition contains.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing cytotoxicity of K8 (Isochaihulactone) on LNCaP cells.

FIG. 2 is a diagram showing cytotoxicity of Z-K8 (Isochaihulactone) on LNCaP (AR-dependent), and PC3 (AR-independent) tumor cell line.

FIGS. 3A and 3B are photographs showing that Z-K8 down regulated AR and PSA (prostate specific antigen) in human cancer cells.

FIG. 4 is a diagram showing that Z-K8 induced G2/M phase arrest and apoptosis in LNACaP cells.

FIG. 5 is a diagram showing that K8 (Isochaihulactone) increased LNCaP cells caspase-3 activity which leads to tumor cell apoptosis.

FIG. 6 is a diagram showing inhibition of human xenografts growth in vivo by isochaihulactone.

FIG. 7 is a diagram showing inhibition of human xenografts growth in vivo by Z-K8.

FIGS. 8A and B are photographs showing that K8 (Isochaihulactone) increased caspase-3 activity and down regulated AR in vivo.

### DESCRIPTION OF THE PREFERRED EMBODIMENT

Unless otherwise stated herein, the terms “a (an)”, “the” or the like used in this specification (especially in the claims) shall be understood to encompass both the singular form and the plural form.

This invention is based on unexpected discoveries that compound isochaihulactone (K8) down regulates androgen receptor and that the Z-from of isochaihulactone (Z-K8) has better antitumor effects than its E-form (E-K8). Besides, the inventors of the present invention also found that isochaihulactone (K8) effectively inhibits the cellular proliferation of androgen dependent or independent prostate cancer cells and thus is useful in the treatment of prostate cancer via a different approach from the conventional hormone therapy.

As described herein, the natural compound isochaihulactone (K8), which can be isolated from the chloroform extract of Nan-Chai-Hu, has been confirmed for its antitumoral effects on lung cancer, hepatoma, breast cancer, colon cancer and urinary bladder cancer both in vitro and in vivo. The target gene of K8 on antitumor effect of lung cancer has also been identified and is mediated Nag by upper regulation and causes tumor apoptosis.

K8's structure was determined. It belongs to lignan which have a chiral-ring center and two racemic forms (the E and Z forms). In order to determine which form has anti-tumor effect, the E form and the Z form have been synthesized. Their anti-tumor effects on prostate cancer have been evaluated. As shown below, the Z-form has tumor cytotoxicity that is more significant than that of the E-form in vitro and in vivo. In addition, this antitumor effect is mediated via down-regulation of androgen receptor (AR) and is also AR dependent.

Prostate cancer is the most common malignancy diagnosed in American males (Greenlee et al., 2000. Cancer statistics: 2000. CA Cancer J. Clin. 50, 7-33.). The detection and treatment of the disease is highly successful when tumors are confined to the prostate with their growth and survival strongly androgen-dependent (AD). However, if left undetected and untreated, many prostate tumors will become androgen-independent (AI) and/or metastatic, and these advanced-stage tumors are associated with high mortality rates. Therefore, it is important to better understand the molecular events associated with the development of AI and/or metastatic prostate cancer to develop more effective therapeutics.

The prostate gland depends upon circulating androgens for proper cellular proliferation, differentiation, and survival, and while prostate cancer cells rely on androgens during early stages of malignant transformation, eventually some transformed cells acquire the ability to grow in their absence (Debes J D and Tindall D J, 2002. The role of androgens and the androgen receptor in prostate cancer. Cancer Lett. 187 (1-2): 1-7). To treat AD prostate tumors, therapies are administered to deplete androgen levels causing normal and transformed prostate cells to undergo apoptosis. Unfortunately, many patients fail this therapy and AI prostate cancer cells emerge to form aggressive AI tumors. In addition, prostate tumors often metastasize to the liver, lungs, and bone, the latter typically causing skeletal fractures and death (Greenlee et al., 2000. Cancer statistics: 2000. CA Cancer J. Clin. 50, 7-33.). During metastasis, tumor cells detach from the primary site, invade the stroma, enter the lymphatic system, implant and grow at distant sites, a process achieved by extracellular matrix (ECM)- and cytoskeletal-remodeling; alterations in cellular adhesion/de-adhesion, proliferation, and angiogenesis; escape from immune-surveillance; and protection from apoptosis. Relatively little is known about the changes in gene expression of androgen receptor that are involved in regulating these processes in prostate cancer.

It has been reported that the AR is the key determinant for the molecular changes required for apoptosis resistance and driving prostate cancer cells from an androgen-dependent to an ADI state (Dehm and Tindall, 2006. Molecular regulation of androgen action in prostate cancer. J Cell Biochem. 99:333-44). Several recent publications suggest that down-regulation of AR expression should therefore be considered as the main strategy for the treatment of ADI prostate cancer (Liao et al., 2005. Small-interfering RNA-induced androgen receptor silencing leads to apoptotic cell death in prostate cancer. Mol Cancer Ther. 4:505-15; Yang et al., 2005. Androgen receptor signaling is required for androgen-sensitive human prostate cancer cell proliferation and survival. Cancer Cell Int. 5:8; Cheng et al., 2006. An oncolytic conditionally replicating adenovirus for hormone-dependent and hormone-independent prostate cancer. Cancer Gene Ther. 13:13-20.).

Nan-Chai-Hu (Chai Hu of the South), the root of Bupleurum scorzonerifolium, is an important Chinese herb in the treatment of influenza, fever, malaria, cancer, and menstrual disorders in China, Japan, and many other parts of the world. It was previously reported that the crude acetone extract of B. scorzonerifolium causes cell cycle arrest of lung adenocarcinoma A549 cells in the G2/M phase, formation of giant cells, and apoptosis (Chen et al., 2005. Requirement for ERK activation in acetone extract identified from Bupleurum scorzonerifolium induced A549 tumor cell apoptosis and keratin 8 phosphorylation. Life Sci. 76:2409-20; Cheng et al., 2005. Phorbol 12-myristate 13-acetate upregulates cyclooxygenase-2 expression in human pulmonary epithelial cells via Ras, Raf-1, ERK, and NF-kappaB, but not p38 MAPK, pathways. Cell Signal. 17:299-310). The acetone extract fraction is further partitioned, and a novel lignan, K8 (isochaihulactone), that has antitumoral activity against A549 cells in vitro and in vivo, was identified (Chen et al., 2006. In vitro and in vivo studies of a novel potential anticancer agent of isochaihulactone on human lung cancer A549 cells. Biochem Pharmacol. 72:308-19.). In previous articles, it has been reported that this compound was isolated from Bursera microphylla (Burseraceae), and had an antitumoral effect (Tomioka et al., 1986. Stereoselective reactions. XII. Synthesis of antitumor-active steganacin analogs, picrosteganol and epipicrosteganol, by selective isomerization. Chem Pharm Bull (Tokyo) 34:1501-1504). These findings indicate that isochaihulactone is a promising new antimitotic anticancer compound with potential for clinical applications. To identify genes that are involved in K8 (isochaihulactone)-induced growth arrest and apoptosis, oligodeoxynucleotide-based microarray screening was used. It was found that several K8 (isochaihulactone)-induced genes are early response genes such as early growth response gene-1 (EGR-1) (also known as NGFI-A, Krox24, TIS8, and Zif/268). EGR-1 is a member of the zinc finger family of transcription factors and plays a role in cell growth and differentiation (Krishnaraju et al., 1995. The zinc finger transcription factor EGR-1 potentiates macrophage differentiation of hematopoietic cells. Mol Cell Biol. 15: 5499-5507; Thiel and Cibelli, 2002. Regulation of life and death by zinc finger transcription factor Egr-1. J Cell Physiol. 193: 287-292.). EGR-1 has been reported to be regulated by numerous growth-regulated genes such as c-myc and transforming growth factor-β (TGF-β), and to inhibit growth. Thrombospondin (TSP) and transforming growth factor β1 (TGF-beta) promote human A549 lung carcinoma cell plasminogen activator inhibitor type 1 (PAI-1) production and stimulate tumor cell attachment in vitro (Albo D. et al., 1994. Thrombospondin (TSP) and Transforming Growth Factor β1 (TGF-beta) Promote Human A549 Lung Carcinoma Cell Plasminogen Activator Inhibitor Type 1 (PAI-1) Production and Stimulate Tumor Cell Attachment in Vitro, Biochem Biophys Res Commun, 203:857-865; Elkon et al., 2004. In silico identification of transcriptional regulators associated with c-Myc. Nucleic Acids Res. 32: 4955-4961.) Induction of EGR-1 expression by antitumorigenic compounds is known to involve members of the family of mitogen-activated protein kinases (MAPKs) or phosphatidylinositol-3-kinase (PI3K)-dependent pathways. For example, induction of EGR-1 expression by the peroxisome proliferator-activated receptor-α (PPAR-α) ligand troglitazone occurs by the ERK phosphorylation pathway rather than by the PPARγ pathway (Baek et al., 2003. Troglitazone, a peroxisome proliferator-activated receptor g (PPARγ) ligand, selectively induces the early growth response-1 gene independently of PPARγ: a novel mechanism for its anti-tumorigenic activity. J Biol Chem 278:5845-5853; Baek et al., 2004. Expression of NAG-1, a transforming growth factor-beta superfamily member, by troglitazone requires the early growth response gene EGR-1. J Biol. Chem. 279: 6883-6892; Baek et al., 2005. Cyclooxygenase inhibitors induce the expression of the tumor suppressor gene EGR-1, which results in the up-regulation of NAG-1, an antitumorigenic protein. Mol. Pharmacol. 67:356-364.). In contrast, PPAR-γ ligands such as 1,1-Bis(3′-indolyl)-1-(p-substituted phenyl)methanes methane compounds induced EGR-1 through a PI3K-dependent pathway, which in turn activated serum-response elements in the EGR-1 promoter (Baek et al., 2003. Troglitazone, a peroxisome proliferator-activated receptor g (PPARγ ligand, selectively induces the early growth response-1 gene independently of PPARγ: a novel mechanism for its anti-tumorigenic activity. J Biol Chem 278:5845-5853).

It was also found that several K8 (isochaihulactone)-induced genes are induced by nonsteroidal anti-inflammatory drugs (NSAIDs). NSAID-activated gene-1 (NAG-1) (also known as MIC-1, GDF-15, placental TGF-β, and PLAB) was highly induced in previous study. NAG-1 is a transforming growth factor-like secreted protein. It was initially characterized as a p53-regulated gene (Baek et al., 2002. Resveratrol enhances the expression of non-steroidal anti-inflammatory drug-activated gene (NAG-1) by increasing the expression of p53. Carcinogenesis 5:425-434.; Bottone et al., 2002. Diallyl disulfide (DADS) induces the antitumorigenic NSAID-activated gene (NAG-1) by a p53-dependent mechanism in human colorectal HCT 116 cells. J Nutr. 132:773-778.; Wilson et al., 2003. Nonsteroidal anti-inflammatory drug-activated gene (NAG-1) is induced by genistein through the expression of p53 in colorectal cancer cells. Int. J Cancer. 105:747-753.). Overexpression of NAG-1 in breast cancer cells both in vitro and in vivo results in growth arrest and apoptosis; similar results were also observed for colon cancer cells (Baek et al., 2004. Epicatechin gallate-induced expression of NAG-1 is associated with growth inhibition and apoptosis in colon cancer cells. Carcinogenesis. 25:2425-2432; Baek et al., 2005. Cyclooxygenase inhibitors induce the expression of the tumor suppressor gene EGR-1, which results in the up-regulation of NAG-1, an antitumorigenic protein. Mol. Pharmacol. 67:356-364; Eling et al., 2006. NSAID activated gene (NAG-1), a modulator of tumorigenesis. J Biochem Mol. Biol. 39:649-655.) and for treatment of prostate cancer cells with purified NAG-1, which induced apoptosis (Liu et al., 2003. Macrophage inhibitory cytokine 1 reduces cell adhesion and induces apoptosis in prostate cancer cells. Cancer Res. 63:5034-5040.). These findings suggest that NAG-1 is linked to apoptosis and that reduced expression of NAG-1 may enhance tumorigenesis.

It is known that the PPARy-dependent activation of NAG-1 by troglitazone is due to induction of EGR-1, which in turn activates NAG-1 (Baek et al., 2004. Epicatechin gallate-induced expression of NAG-1 is associated with growth inhibition and apoptosis in colon cancer cells. Carcinogenesis. 25:2425-2432; Baek et al., 2004. Expression of NAG-1, a transforming growth factor-β superfamily member, by troglitazone requires the early growth response gene EGR-1. J Biol. Chem. 279:6883-6892; Yamaguchi et al., 2006. A novel peroxisome proliferator-activated receptor γ ligand, MCC-555, induces apoptosis via posttranscriptional regulation of NAG-1 in colorectal cancer cells. Mol Cancer Ther 5:1352-1361). Like troglitazone, the PPARγ-active 1,1-bis(3′-indolyl)-1-(p-substituted phenyl)methanes also induce EGR-1, which in turn interacts with proximal (GC-rich) EGR-1 motifs in the NAG-1 promoter (Chintharlapalli et al., 2006. 1,1-Bis(3′-indolyl)-1-(p-substituted phenyl)methanes inhibit colon cancer cell and tumor growth through PPARγ-dependent and PPARγ-independent pathways. Mol Cancer Ther. 5:1362-1370 and Chintharlapalli et al., 2005. 1,1-Bis(3′-indolyl)-1-(p-substituted phenyl)methanes are peroxisome proliferatoractivated vreceptor-γ agonists but decrease HCT-116 colon cancer cell survival through receptor-independent activation of early growth response-1 and nonsteroidal anti-inflammatory drug-activated gene-1. Mol. Pharmacol. 66:1783-1792.). This reaction represents a pathway for induction of EGR-1 and NAG-1, and these responses contribute to the in also a major ingredient of traditional Chinese medicine for induction of growth inhibition and apoptosis by antitumoral compounds in cancer cells. Because NAD is also named PDF (prostate differentiated factor), this urge the inventors of the present invention to investigate the antitumor effect on prostate cancer.

### Treatment Methods

The invention also features methods for treating in a subject a cellular proliferative disorder (e.g., cancer). A cellular proliferative disorder refers to a disorder characterized by uncontrolled, autonomous cell growth, including malignant and non-malignant growth. Examples of this disorder include oral cancer, gastric cancer, colon cancer, breast cancer, prostate cancer, hepatocellular carcinoma, melanoma, lung cancer, glioblastoma, brain tumor, hematopoeitic malignancies, retinoblastoma, renal cell carcinoma, head and neck cancer, cervical cancer, pancreatic cancer, esophageal cancer, and squama cell carcinoma.

Specifically, this invention provides a method of inhibiting the cellular proliferation of at least one selected from the group consisting of androgen dependent prostate cancer cells, androgen independent prostate cancer cells, oral cancer cells, gastric cancer cells, and liver cancer cells in a subject, comprising administrating to the subject an effective amount of an active component selected from the group consisting of Z-K8 of the formula (I), E-K8 of the formula (II), a pharmaceutically acceptable salt of Z-K8 or E-K8, a pharmaceutically acceptable ester of Z-K8 or E-K8, and combinations thereof:

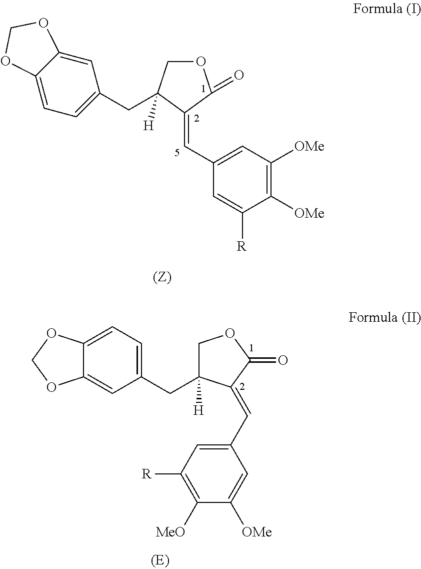

wherein R is H, an alkoxy, or an aryl.

Preferably, in formulae (I) and (II), R is H, a C₁-C₆ alkoxy, or a C₆-C₂₀ aryl, and more preferably, R is methoxy. Because there is a chiral center (marked with *) in the structure of Z-K8 as well as E-K8, these compounds have enantiomers. The active components in the present invention encompass these enantiomers.

In the examples below, most notably human prostate cancer cell line LanCap and PC3 with androgen dependent and independent cell line respectively were chosen to study E-K8 (E-isochaihulactone) and Z-K8 (Z-isochaihulactone) to determine their antitumor potency. First, E-K8 and Z-K8 were synthesized by a novel method as described below. Subsequently, their anti-tumor effects on prostate cancer were evaluated. The results show that the Z-form has tumor cytotoxicity that is more significant than that of E-form in vitro and in vivo. Thus, in the method of the present invention, the active component is preferably selected from the group consisting of Z-K8, a pharmaceutically acceptable salt of Z-K8, a pharmaceutically acceptable ester of Z-K8, and combinations thereof. The active component in the present invention may be administrated as a medicament.

In addition, this antitumor effect was found to mediate via the down regulation of androgen receptor(AR) and was AR dependent. On the other hand, Z-K8 may also inhibit the growth of androgen independent cell line PC3, and this antitumor effect was found to mediate via the promotion of apoptosis of prostate cancer cells by activating caspase-3 and was AR independent. Thus, the method according to the present invention is useful in treating androgen dependent or independent prostate cancer.

Prostate specific antigen (PSA) is a tumor marker of prostate cancer, which can be used for the diagnosis and clinical staging of prostate cancer, as well as for tracking the treatment effect. Z-K8 may inhibit the expression of PSA, showing the anti-prostate cancer cell effect thereof.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

A subject to be treated for a cellular proliferative disorder can be identified by standard diagnosing techniques for the disorder. Optionally, the subject can then be examined for the gene expression or activity level of the AR gene or polypeptide by methods known in the art or described below. If the gene expression or activity level is higher in a sample from the subject than that in a sample from a noimal person, the subject is a candidate for treatment with an effective amount of a compound described herein.

“Treating” refers to administration of a compound to a subject, who/which has a cellular proliferative disorder (e.g., cancer), with the purpose to cure, alleviate, relieve, remedy, ameliorate, or delay the onset of the disorder, the symptom of the disorder, the disease state secondary to the disorder, or the predisposition toward the disorder. An “effective amount” refers to an amount of the compound that is capable of producing a medically desirable result, e.g., as described above, in a treated subject. The treatment method can be performed in vivo or ex vivo, alone or in conjunction with other drugs or therapy.

In an in vivo approach, a compound is administered to a subject. Generally, the compound is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg-body weight. For example, when Z-K8 is used in the treatment of prostate cancer in a human, the dosage of Z-K8 is about 1 mg/kg-body weight to about 400 mg/kg-body weight per day, preferably about 15 mg/kg-body weight to about 35 mg/kg-body weight per day, based on the total weight of Z-K8. Variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Examples of compounds that can be used to treat a cellular proliferative disorder include isochaihulactone (K8), in particular, the Z-form. The aforementioned compounds can be synthesized using methods known in the art or be prepared using the methods descried below. As is well known in the art, the dosage for a patient depends upon various factors as described above. Routes of administration can be any of those listed above.

Also within the scope of the invention is a packaged product including a container, an effective amount of one of the above-described compound and a legend associated with the container and indicating administration of the compound for treating a subject suffering from or being at risk for developing the disorder mentioned above. The compound can be admixed with a pharmaceutically acceptable carrier, including a solvent, a dispersion medium, a coating, an antibacterial and antifungal agent, and an isotonic and absorption-delaying agent.

The compound can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the compound with a solid carrier and a lubricant. Examples of solid carriers include starch and sugar bentonite. The compound can also be administered in a form of a hard shell tablet or a capsule containing a binder, e.g., lactose or mannitol, a conventional filler, and a tableting agent. The compound can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of the compound can be evaluated both in vitro and in vivo. For example, the compound can be tested for its ability to arrest cell growth or induce apoptosis in vitro. For in vivo studies, the compound can be injected into an animal (e.g., an animal model) and its effects on cell growth or apoptosis are then accessed. Based on the results, an appropriate dosage range and administration route can be determined.

### Compositions

Within the scope of this invention is a composition that contains a suitable carrier and one or more of the compounds described above. The composition can be a pharmaceutical composition that contains a pharmaceutically acceptable carrier, a dietary composition that contains a dietarily acceptable suitable carrier, or a cosmetic composition that contains a cosmetically acceptable carrier.

Depending on the kind of the composition, a carrier may be a suitable dietary carrier or a pharmaceutically acceptable carrier. Examples of a pharmaceutically acceptable carrier include, but are not limited to, biocompatible vehicles, adjuvants, additives, and diluents to achieve a composition usable as a dosage form.

A “pharmaceutically acceptable carrier,” after administered to or upon a subject, does not cause undesirable physiological effects. The carrier in the pharmaceutical composition must be “acceptable” also in the sense that it is compatible with the active ingredient and, preferably, capable of stabilizing it. One or more solubilizing agents can be utilized as pharmaceutical carriers for delivery of an active compound. Examples of other carriers include colloidal silicon oxide, magnesium stearate, cellulose, sodium lauryl sulfate, and D&C Yellow # 10.

The above-described composition, in any of the forms described above, can be used for treating cellular proliferative disorders and inflammation-related disorders.

An “effective amount” refers to the amount of an active compound that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having an active compound can also be administered in the form of suppositories for rectal administration.

A topical composition contains a safe and effective amount of a dermatologically acceptable carrier suitable for application to the skin. Generally, a topical composition can be solid, semi-solid, cream, or liquid. It may be a cosmetic or dermatologic product in the form of an ointment, lotion, foam, cream, gel, or solution. Details about dermatologically acceptable carriers are provided below.

A composition of the present invention may be used alone or in combination with other biologically active ingredients. Alone or in combination with other active ingredients, it may be administered to a subject in a single dose or multiple doses over a period of time. Various administration patterns will be apparent to those skilled in the art. The dosage ranges for the administration of the composition are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the present invention to be used for an intended purpose.

Within the scope of this invention is a dietary composition. Examples of a dietary composition of the present invention include an active compound described above. The composition also includes, but is not limited to, foods, food additives, nutritional supplements, and pharmaceutical preparations. It may be in the form of tablets, suspensions, implants, solutions, emulsions, capsules, powders, syrups, liquid compositions, ointments, lotions, creams, pastes, gels, or the like.

As a dietary supplement, additional nutrients, such as minerals or amino acids, may be included. A dietary composition can also be a drink or food product. As used herein, the terms “drink” and “food” broadly refer to any kinds of liquid and solid/semi-solid materials, respectively, that are used for nourishing an animal, and for sustaining normal or accelerated growth of an animal including a human. Examples of the drink product include, but are not limited to, tea-based beverages, juice, coffee, and milk. Examples of the food product include jelly, cookies, cereals, chocolates, snack bars, herbal extracts, dairy products (e.g., ice cream, and yogurt), soy bean product (e.g., tofu), and rice products.

Also within the scope of this invention is a cosmetic composition that contains an active compound described above. This composition contains a safe and effective amount of a dermatologically acceptable carrier that is suitable for topical application to the skin. It enables an active compound and optional component to be delivered to the skin at an appropriate concentration(s). The carrier can thus act as a diluent, dispersant, solvent, or the like to ensure that the active materials are applied to and distributed evenly over the selected target at an appropriate concentration. The carrier can be solid, semi-solid, or liquid. Preferably, it is in the form of a lotion, a cream, or a gel, in particular one that has a sufficient thickness or yield point to prevent the active materials from sedimenting. The carrier can be inert or possess dermatological benefits of its own. It should also be physically and chemically compatible with the active components described herein, and should not unduly impair stability, efficacy, or other use benefits associated with the composition.

The type of carrier utilized in the cosmetic composition depends on the type of product form of the composition. A cosmetic composition may be made into a wide variety of product forms such as those known in the art. These include, but are not limited to, lotions, creams, gels, sticks, sprays, ointments, pastes, and mousses. These product forms may comprise several types of carriers including, but not limited to, solutions, aerosols, emulsions, gels, solids, and liposomes.

### Novel Method for Preparing Z-K8 and E-K8

Currently, isochaihulactone is mainly isolated from Nan-Chai-Hu (Bupleurum scorzonerifolium), which requires complicated extraction and purification procedures. The methods for isolation of isochaihulactone can be seen in, for instance, U.S. Patent Application Nos. 2005/0013879 and 2006/0079575, which are incorporated hereinto by reference. The conventional isolation methods not only are restricted by the source of Nan-Chai-Hu, but also involve complicated purification procedures, and thus are not suitable for mass production.

The present invention provides a chemical synthesis method for manufacturing the racemic mixtures of isochaihulactone. This method does not require complicated extraction or purification procedures, and is not restricted by the source of Nan-Chai-Hu. Furthermore, the method of the present invention avoids the drawbacks in the conventional methods that the end product may contain significant amount of impurities due to the isolation of isochaihulactone from mixtures comprising numerous components.

Z-K8 and E-K8 may be manufactured by a novel method comprising:

(a) providing a first solution, which is a dichloromethane solution containing a first compound of the following formula (III) and anhydrous triethylamine,

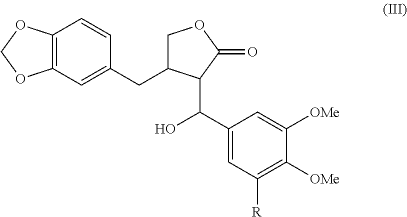

wherein, R is H, methoxy, or an aryl;

(b) providing a second solution, which is an anhydrous triethylamine solution containing methanesulfonyl chloride;

(c) mixing the first solution and the second solution under an inert gas atmosphere to provide a first reacting mixture;

(d) adding the first reacting mixture into a hydrochloric acid to provide a second reacting mixture, and extracting the second reacting mixture with dichloromethane or ethyl estate and collecting organic phase to provide a first extract;

(e) drying the first extract to provide a residue; and

(f) dissolving the residue and a second compound into acetonitrile to carry out an reaction to obtain the racemic mixture, the second compound is selected from a group consisting of 1,8-diazabicyclo[5.4.0]undec-7-ene (DBU), 1,4-diazabicyclo[2.2.2]octane (DABCO) and a combination thereof.

There is no particular limitation for the first compound of the formula (III) in step (a). In one embodiment of the method of the present invention, mixtures of erythro and threo α-[hydroxyl(3′,4′,5′-trimethoxyphenyl)methyl]-β-(3′,4′-methylenedioxybenzyl)-γ-butyrolactones are used as the first compound, which can be provided by reacting piperonal with dibutyl succinate. First, piperonal is mixed with dibutyl succinate in a proper proportion (e.g., in a molar ratio of about 1:1) to form 4-(3′,4′-methylenedioxyphenyl)-3-ethoxycarbonyl-3-butenoic acid. Then, a reduction reaction is carried out to reduce 4-(3′,4′-methylenedioxyphenyl)-3-ethoxycarbonyl-3-butenoic acid to 4-(3′,4′-methylene dioxyphenyl)-3-ethoxycarbonyl-3-butanoic acid. Herein, without being limited by theories, the reduction reaction can be performed by any conventional approaches, such as the hydrogen gas catalysis method (which can be seen in, for instance, Morimoto et al., 2005. Lipase-Catalyzed Esterification of a (+/−)-2,3-Di(Arylmethyl)-1,4-butanediol and Its Application to the Synthesis of (S.S)-(+)-Hinokinin, Synthetic Communications, 35:857-865; and Kirlj et al., 2007. Conformational Study of (8α,8′β)-Bis(substituted phenyl)-Lignano-9,9′-lactones by Means of Combined Computational, Database Mining, NMR, and Chemometric Approaches, J. Phys. Chem. A, 111, 6316-6333, which are incorporated hereinto by reference) or the metal reduction method. Preferably, the metal reduction method is adopted to obtain a high yield and reduce reaction time. Then, 4-(3′,4′-methylenedioxyphenyl)-3-ethoxycarbonyl-3-butanoic acid is subjected to an esterification reaction to form β-(3′,4′-methylenedioxybenzyl)-γ-butyrolactones. The esterification reaction can be carried out by adopting any conventional approaches. For example, the carboxyl group of 4-(3′,4′-methylenedioxyphenyl)-3-ethoxycarbonyl-3-butanoic acid can be converted to potassium salts, which then reacted with calcium borohydride. This method can be seen in, for instance, Morimoto et al., 2005. Lipase-Catalyzed Esterification of a (+/−)-2,3-Di(Arylmethyl)-1,4-butanediol and Its Application to the Synthesis of (S.S)-(+)-Hinokinin, Synthetic Communications, 35:857-865. Eventually, β-(3′,4′-methylenedioxybenzyl)-γ-butyrolactones is subjected to a condensation reaction with 3,4,5-trimethoxybenzaldehyde to provide the mixtures of erythro and threo α-[hydroxyl(3′,4′,5′-trimethoxyphenyl)methyl]-β-(3′,4′-methylenedioxybenzyl)-γ-butyrolactones.

Step (c) is carried out under the presence of an inert gas, and the inert gas can be selected by persons with ordinary knowledge in this field depending on the demands. For instance, the inert gas can be selected from the group consisting of nitrogen gas, helium gas, argon gas, and combinations thereof. Besides, in step (c), the first solution and the second solution are preferably mixed under about −10° C. to 10° C., more preferably under about −5° C. to 5° C., to provide a first reacting mixture. For example, the first solution and the second solution can be mixed in an ice-bath under the nitrogen atmosphere.

In step (d), the first reacting mixture obtained in step (c) is poured into a hydrochloric acid solution to provide a second reacting mixture. Then, the second reacting mixture is extracted with dichloromethane or ethyl acetate, and the organic phase is collected to provide a first extract. In step (d), the extraction procedure is preferably repeated several times (e.g., repeated two to four times), and the organic phase obtained in each extraction procedure is combined to provide the first extract. In one embodiment of the present invention, the second reacting mixture is extracted with dichloromethane for three times.

Thereafter, in step (e), the first extract from step (d) is dried to provide a residue. Step (e) can be carried out with any suitable conventional approaches. For example, the first extract can be dried with an approach selected from the group consisting of baking, air drying, and air extraction drying. In one embodiment of the present invention, the first extract is dried by air extraction drying to avoid possible product deterioration due to baking under high temperature or long-period air drying.

Preferably, after the first extract is obtained and prior to step (e), the first extract is washed with a sodium bicarbonate solution first and then a sodium chloride solution to remove impurities soluble in aqueous phase in the first extract. For example, in one embodiment of the present invention, the first extract is washed with a cold (about 0 to 5° C.) saturated sodium bicarbonate solution first and then a cold saturated sodium chloride solution. The aforesaid washing procedure can also be optionally operated repeatedly.

Afterwards, in step (f), the residue obtained in step (e), along with a second compound, is dissolved into acetonitrile to obtain the desired racemic mixtures of isochaihulactone. There is no a particular limit for the mixing proportion of the residue and the second compound. The second compound can be 1,8-diazabicyclo[5.4.0]undec-7-ene (DBU), 1,4-diaza-bicyclo[2.2.2]octane (DABCO), and a combination thereof. DBU is preferable. Generally, step (f) is carried out under about 40 to 60° C. (e.g., 50° C.). Optionally, during the reaction of step (f), an assistant approach, such as agitating, can be adopted to elevate reaction efficiency.

Optionally, after step (f), a purification operation is further performed to obtain the racemic mixtures of isochaihulactone from the reaction product in step (f). Herein, after the completion of the reaction of step (f), the reaction product of step (f) is purified to provide the racemic mixtures of isochaihulactone in accordance with the aforesaid extraction and drying operation of obtaining the residue of step (e) from the second reacting mixture of step (d), along with separation and optional washing (removing impurities) operation.

For example, after the completion of the reaction of step (f), the reaction product of step (f) is cooled to room temperature, and a suitable amount of ice water is added thereto with agitation to provide a mixture. Thereafter, the mixture is extracted with dichloromethane or ethyl acetate. There is no particular limit for the number of the extraction times, but preferably, the extraction is repeated several times (e.g., two to four times) to ensure the extraction effect. In one embodiment of the present invention, the fourth reacting mixture is extracted with dichloromethane repeatedly for three times. After the extraction procedure, all the collected organic phase is combined to provide a second extract, and then the second extract is further washed by cold water and a saturated sodium chloride solution to remove undesired impurities in the second extract. Similarly, there is no a particular limit for the number of washing times. Then, the second extract is dried with the aforesaid physical or chemical drying approaches (e.g., using anhydrous magnesium sulfate) to obtain a crude product of the racemic mixtures of isochaihulactone. Finally, a purification step is optionally conducted to individually isolate the racemic mixtures of isochaihulactone from the obtained crude product. The purification step can be carried out by any suitable conventional approaches, such as column chromatography, recrystallization, etc. In one embodiment of the present invention, the purification step is carried out by silica gel column chromatography using ethyl actetate/n-hexane as eluant.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### Example I

### Chemicals Synthesizing (K8 (isochaihulactone) E and Z Racemic Form)

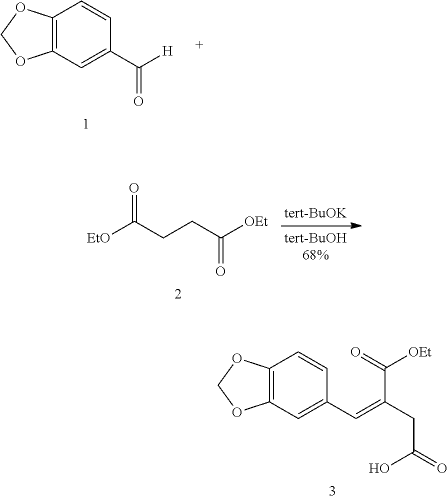

### 4-(3′,4′-Methylenedioxyphenyl)-3-ethoxycarbonyl-3-butenoic Acid (3).^{1,2}

To an oven-dried 300 mL three-neck flask equipped with addition funnel, stirring bar and a reflux condenser protected with a calcium chloride tube was added potassium tert-butoxide (8.63 g, 76.92 mmol) and 80 mL of anhydrous tert-butanol. A solution of piperonal (1) (10.98 g, 73.13 mmol) and diethyl succinate (2) (13.40 g, 76.92 mmol) in anhydrous tert-butanol (80 mL) was added dropwise over 15 min at refluxing temperature under nitrogen atmosphere. The reaction mixture was stirred for another 2 h, and monitored by TLC until the disappearance of piperonal. The cooled mixture was acidified with 3 M HCl (ca. 26.5 mL) at 0° C. and the solvent was evaporated under reduced pressure. The resulting mixture was then diluted with 10 mL of water and 80 mL of dichloromethane. The aqueous layer was extracted with dichloromethane (2×20 mL) and the combined organic extracts were washed with water (2×10 mL), brine (10 mL) and dried over anhydrous magnesium sulfate. After removal of solvent the light yellow residue was purified by column chromatography through silica gel, eluting with gradient n-hexane/ethyl acetate mixture to afford the desired unsaturated monoester 3 (13.84 g, 68%) as pale yellow solid. mp 112˜113° C., ¹H NMR (CDCl₃, 300 MHz) δ 7.81 (s, 1H), 6.92-6.84 (m, 3H), 6.00 (s, 2H), 4.28 (q, 2H, J=7.2 Hz), 3.58 (s, 2H), 1.33 (t, 3H, J=7.2 Hz); ¹³C NMR (CDCl₃) δ 176.78, 167.61, 148.38, 147.96, 142.07, 128.61, 123.98, 123.93, 109.09, 108.60, 101.41, 61.35, 33.67, 14.17.

1. Morimoto et al., 2005. Syn. Comm. 35, 857-865.

2. Cow et al., 2000. Can. J. Chem., 78(5), 553-561. ( )

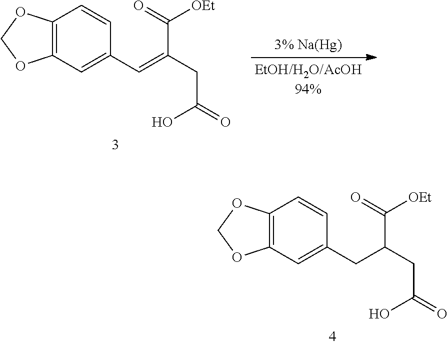

### 4-(3′,4′Methylenedioxyphenyl)-3-ethoxycarbonylbutanoic Acid (4)

A 3% sodium amalgam was prepared from sodium (3.22 g, 0.14 mol) and mercury (106.7 g) in a three-neck flask under nitrogen. Dropwise addition of a solution containing monoester 3 (10.2 g, 36.6 mmol), 95% ethanol (100 mL), acetic acid (10 mL) and water (10 mL) was carried out over 2 h under nitrogen at 0° C. After the mixture was warmed to room temperature, the mercury and insoluble materials were removed by decantation and filtration. The reaction mixture was acidified by adding 5% HCl and then concentrated by evaporation under reduced pressure. The resulting residue was dissolved in dichloromethane (100 mL) and washed with water (2×10 mL) and dried over anhydrous magnesium sulfate. The residue after removal of the solvent was purified by flash chromatography (15% ethyl acetate in hexanes) to give 4 (9.65 g, 94%) as a pale yellow oil. ¹H NMR (CDCl₃, 300 MHz) δ 6.71 (d, 1H, J=7.8 Hz), 6.65 (d, 1H, J=1.5 Hz), 6.60 (dd, 1H, J=1.5, 7.8 Hz), 5.92 (s, 2H), 4.13 (q, 2H, J=7.2 Hz), 3.00˜2.96 (m, 2H), 2.73-2.65 (m, 2H), 2.43 (dd, 1H, J=4.8, 17 Hz), 1.20 (t, 3H, J=7.2 Hz); ¹³C NMR (CDCl₃) δ 177.76, 173.96, 147.70, 146.31, 131.65, 122.05, 109.25, 108.21, 100.90, 60.86, 43.02, 37.30, 34.71, 14.07.

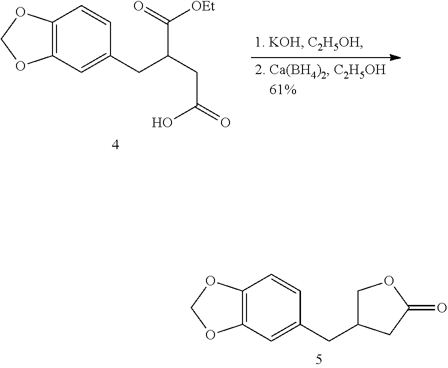

### β-(3′,4′-Methylenedioxybenzyl)-γ-butyrolactone (5)³⁻⁵

A solution of 4 (4.02 g, 14.3 mmol) and one drop of 1% phenolphthalein in ethanol was slowly added with 5% potassium hydroxide ethanolic solution at room temperature until the red color maintained for 1 min. The solvent was evaporated under reduced pressure and dried in vacuum to give potassium salt of 4. Another three-neck flask containing anhydrous ethanol (100 mL) was added with calcium chloride (3.81 g, 34.1 mmol) under nitrogen at 0° C. The resulting solution was stirred until the solid of calcium chloride completely dissolved and then followed by addition of a solution of sodium borohydride (2.05 g, 54.45 mmol) in anhydrous ethanol (50 mL) at −20° C. After stirring at −20° C. for 30 min., a solution of potassium salt of 4 in anhydrous ethanol (40 mL) was added into the calcium borohydride solution at −20° C. and stirred for another 4 h. The reaction solution was slowly warmed to 0° C. and then diluted with water (20 mL). The milky solution was acidified with 3 M HCl and clarified. After evaporation of ethanol the aqueous residue was diluted with water (30 mL) and extracted with dichloromethane (3×30 mL). The combined extracts was washed with water (10 mL), dried over magnesium sulfate and concentrated. The residue was purified by chromatography (8% ethyl acetate in hexanes) to afford butyrolactone 5 (1.92 g, 61%)¹ as a colorless oil. ¹H NMR (CDCl₃, 300 MHz) δ 6.74 (d, 1H, J=7.5 Hz), 6.63 (d, 1H, J=1.5 Hz), 6.60 (dd, 1H, J=1.5, 7.8 Hz), 5.94 (s, 2H), 4.32 (dd, 1H, J=6.9, 9.0 Hz), 4.01 (dd, 1H, J=6.0, 9.3 Hz), 2.74˜2.82 (m, 1H), 2.55-2.69 (m, 3H), 2.26 (dd, 1H, J=6.9, 17.4 Hz); ¹³C(CDCl₃) δ 176.76, 147.90, 146.36, 131.85, 121.60, 108.81, 108.42, 101.00, 72.50, 38.63, 37.29, 34.10.

In a reaction, the residue before purification was subjected to intramolecular dehydration in the Dean-Stark apparatus containing a solution of benzene and catalytic amount of toluenesulfonic acid monohydrate. The isolated yield of butyrolactone 5 was increased to 78%.

3. Minami et al., 1986. Chem. Lett., 1229-1232.

4. Honda et al., 1994. J. Chem. Soc., Perkin Trans. I., 1043.

5. Kiralj et al., J. Phys. Chem. A, 111, 6316-6333, (2007),

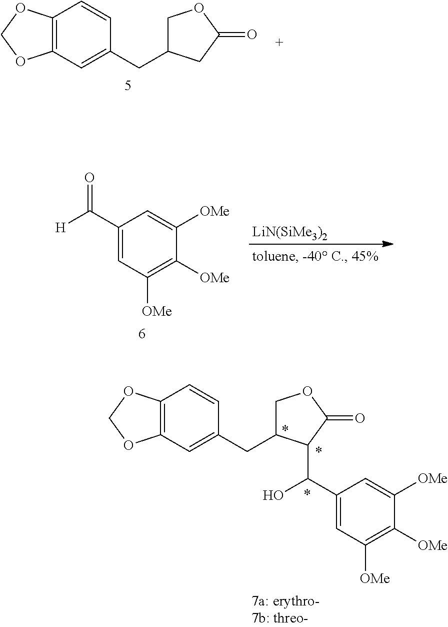

### Mixture of α-[hydroxyl(3′,4′,5′-trimethoxyphenyl)methyl]-β-(3′,4′-methylenedioxybenzyl)-γ-butyrolactones (7a and 7b)^{6,7}

Prepare a three-neck flask equipped with two addition funnel and a stirring bar. A solution of hexamethyldisilazane (7.62 g, 47.2 mmol) in dry toluene (20 mL) was stirred with n-butyllithium (2.5 M in hexane, 20 mL) at 0° C. under nitrogen for 15 min. A solution of γ-butyrolactone 5 (2.40 g, 10.9 mmol) in toluene (10 mL) was added from the addition funnel, and stirring was continued for 5 min. at −40° C. Subsequently, a solution of 3,4,5-trimethoxybenzaldehyde (6) (2.32 g, 11.8 mmol) in toluene (25 mL) was added from the second funnel and the reaction mixture was stirred at −40° C. for 0.5 h and −10° C. for 2 h. The reaction mixture was quenched with 10% aqueous ammonium chloride (10 mL) and then extracted with dichloromethane (3×30 mL). The combined organic layers were washed with 2% HCl (10 mL), cold water (10 mL), saturated sodium bicarbonate and dried over anhydrous magnesium sulfate. After removal of the solvent, the residue was purified by chromatography (0.2% methanol in dichloromethane) afforded a mixture of diastereomers 7a and 7b. (2.04 g, 45%) as a yellow oil in a ratio of 40:60 from the integration of the signals at δ5.27 (erythro-, 1H, m, unresolved)) and δ 4.80 (threo-; 1H, d. J=8.1 Hz).

6. Tomioka et al., 1985. Chem. Pharm. Bull., 33(10), 4333-4337.

7. Yamauchi et al., 1999. Biosci. Biotechnol. Biochem., 63(8), 1453-1462.

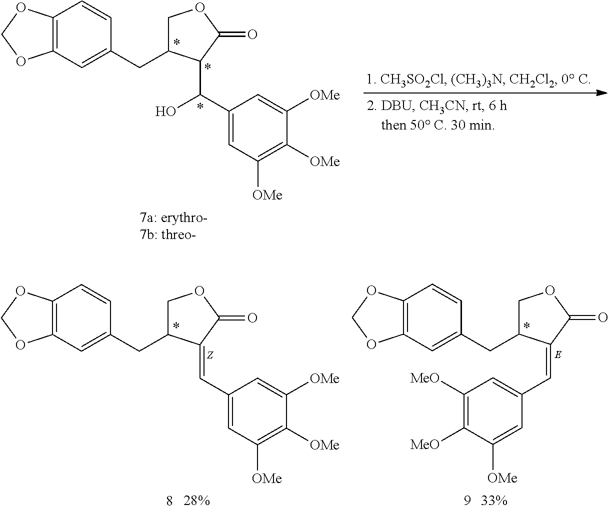

### Racemic (Z)-β-(3′,4′-Methylenedioxybenzyl)-α-(3′,4′,5′-trimethoxybenzylidene)-γ-butyrolactone (8) and Racemic (E)-Form Isomers (9).⁸

A mixture of diasteroemeric carbinols 7a, 7b (0.64 g, 1.53 mmol) and anhydrous triethylamine (0.31 g, 3.06 mmol) in dichloromethane (10 mL) solution was added dropwise to a stirred solution of methanesulfonyl chloride (3.88 g, 20.35 mmol) in anhydrous triethylamine (10 mL) at 0° C. under an atmosphere of nitrogen. The reaction mixture was stirred for 1 h in an ice-bath. The ice-bath was removed and stirring was continued at room temperature for 2 h. The reaction mixture was poured into cooled 10% HCl solution and extracted with dichloromethane (3×20 mL). The organic extracts were washed with cold NaHCO₃ solution (2×10 mL) and brine (15 mL) and dried. After removal of the solvent, the residue was dissolved in acetonitrile (20 mL) and added with 1,8-diazabicyclo[5,4,0]undec-7-ene (DBU) (0.24 g, 1.57 mmol). The reaction mixture was stirred at room temperature for 6 h and 50° C. for 30 min. It was then cooled and diluted with ice-water (10 mL), and the solution was extracted with dichloromethane (3×10 mL). The extracts were washed with water and saturated brine and dried. Purification of the crude product by silica gel chromatography (10% ethyl acetate in hexanes) gave racemic (Z)-form 8 (0.17 g, 28%) and (E)-form 9 (0.20 g, 33%) as light yellow solids.

Racemic (Z)-form 8: mp 131-132° C. (hexanes/ethyl acetate) (lit.₈. (S)-(Z)-form 137-138° C.), R_{f}0.46 (30% ethyl acetate in n-hexane), IR(KBr) 2962, 2925, 2898, 1745, 1632, 1582, 1502, 1489, 1466, 1244, 1154, 1080, 1028 and 923 cm⁻¹. ¹H NMR (CDCl₃, 300 MHz) δ 7.25 (s, 2H), 6.76 (d, 1H, J=8.1 Hz), 6.70 (d, 1H, J=1.8 Hz), 6.62 (dd, 1H, J=8.1, 1.8 Hz), 6.61 (d, 1H, J=1.5 Hz), 5.96 (d, 1H, J=1.5 Hz), 5.95 (d, 1H, J=1.5 Hz), 4.34 (dd, 1H, J=7.2, 9.0 Hz), 4.17 (dd, 1H, J=3.9, 9.0 Hz), 3.89 (s, 9H), 3.29-3.33 (m, 1H), 2.94 (dd, 1H, J=6.9, 13.7 Hz), 2.80 (dd, 1H, J=9, 13.4 Hz); ¹³C(CDCl₃) δ 169.26, 152.63, 147.95, 146.49, 140.59, 139.60, 131.31, 128.83, 126.36, 122.29, 109.30, 108.70, 108.40, 101.06, 69.83, 60.92, 56.21, 44.46, 40.75.

Racemic (E)-form 9: mp 111-112° C. (hexanes/ethyl acetate), R_{f}0.33 (30% ethyl acetate in n-hexane), IR(KBr) 2925, 2851, 1747, 1647, 1580, 1504, 1425, 1336, 1253, 1190, 1129, 1075, 1035, 928 cm⁻¹; ¹H NMR (CDCl₃, 300 MHz) δ 7.51 (d, 1H, J=2.1 Hz), 6.77 (s, 2H), 6.69 (dd, 1H J=0.6 Hz, 7.5 Hz), 6.61 (s, 1H, unresolved), 6.59 (d, 1H, J=7.8 Hz), 5.93 (d, 1H, J=1.2 Hz), 5.92 (d, 1H, J=1.2 Hz), 4.30 (dd, 1H, J=3.0, 8.6 Hz), 4.27 (dd, 1H, J=2.4 Hz, 6.0 Hz), 3.90 (s, 3H), 3.89 (s, 6H), 3.80-3.84 (m, 1H), 3.02 (dd, 1H, J=4.5, 14.4 Hz), 2.65 (dd, 1H, J=9.9, 14.4 Hz); ¹³C(CDCl₃) δ 172.31, 153.36, 147.94, 146.56, 139.81, 137.68, 131.19, 129.40, 127.01, 121.84, 108.99, 108.43, 107.30, 101.09, 69.68, 61.00, 56.25, 39.50, 37.74.

8. Jeong et al., 2007. Biol. Pharm. Bull. 30(7), 1340-1343.

### Example II

### Bioassays

### Material and Methods

1. Chemicals and Reagents

Isochaihulactone was dissolved in DMSO to a concentration of 50 mM and stored in 20° C. as a master stock solution. Dimethyl sulfoxide (DMSO)), 3-(4,5-dimethylthiazol-2-yl)-2,5-diphenyltetrazolium bromide (MTT), were purchased from Sigma Chemical Co. (St. Louis, Mo., USA). RPMI 1640 medium, Eagle's minimumessential medium, fetal bovine serum (FBS), penicillin, streptomycin, trypsin/EDTA, NuPAGE Bis-Tris Electrophoresis System (pre-cast polyacrylamide mini-gel) were purchased from Invitrogen (Carlsbad, Calif., USA). Mycoplasma Removal Reagent was from Dainippon Pharmaceutical Co. (Osaka, Japan).

2 Cell Lines and Culture

The LNCaP (androgen-sensitive), PC3 (androgen in-dependent), human prostate cancer and normal human fibroblast 3T3 were obtained from American Type Culture Collection (Manassas, Va.) were maintained with RPMI 1640 medium containing 10% FBS and 100 ng/ml each of penicillin and streptomycin at 37.8° C. in a humidified atmosphere with 5% CO₂. All cultures were free of mycoplasma.

3 Growth Inhibition Assay

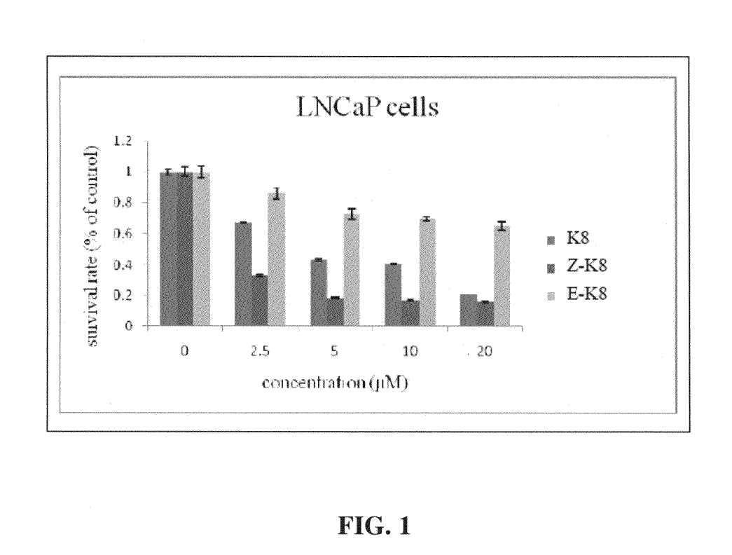

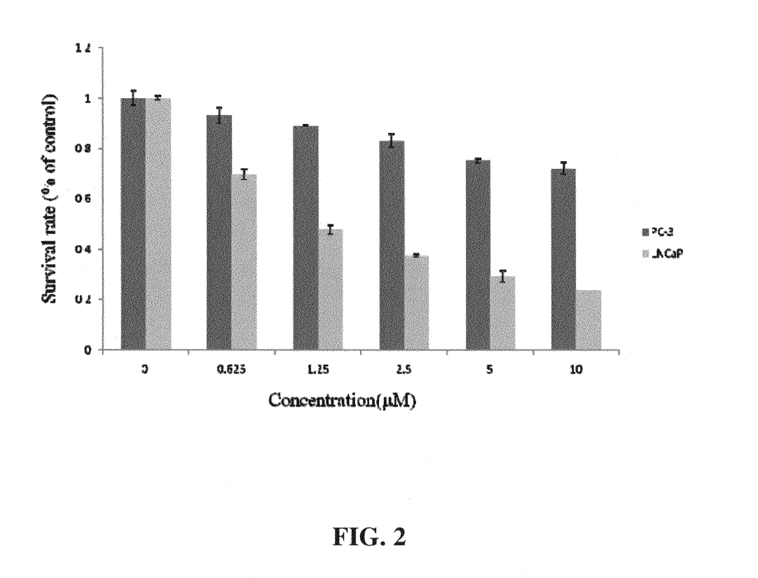

The viability of the cells after treated with various chemicals was evaluated using MTT assay preformed in triplicate (Mosmann T. 1983. Rapid colorimetric assay for cellular growth and survival: application to proliferation and cytotoxicity assays”. Journal of immunological methods. 65 (1-2): 55-63). Briefly, the cancer cells (5×10³) were incubated in 96-well plates containing 200 ml of the culture medium. Cells were permitted to adhere for 12-18 h then washed with phosphate buffered saline (PBS). Solutions were always prepared fresh by dissolving 0.2% DMSO or K8 in culture medium and added to various kind of tumor. After 48 h of exposure, K8 containing medium was removed, washed with PBS and replaced by fresh medium. The cells in each well were then incubated in culture medium with 500 mg/ml MTT for 4 h. After the medium were removed, 200 ml of DMSO and 25 ml of glycine buffer (0.1 M glycine, 0.1 M NaCl, pH 10.5) were added to each well. Absorbance at 570 nm of the maximum was detected by a PowerWave X Microplate ELISA Reader (Bio-TeK Instruments, Winoski, Vt.). The absorbance for DMSO treated cells was considered as 100%. The results were determined by three independent experiments. The results are shown in Tables 1 and 2 and FIGS. 1 and 2.

Besides, the above assay was also conducted on gastric cancer cell line NCI-N87, liver cancer cell line HepG2 and J5, lung cancer cell line A549, and oral cancer cell line OECM-1. The results are shown in Table 3.

4. RT-PCR Analysis.

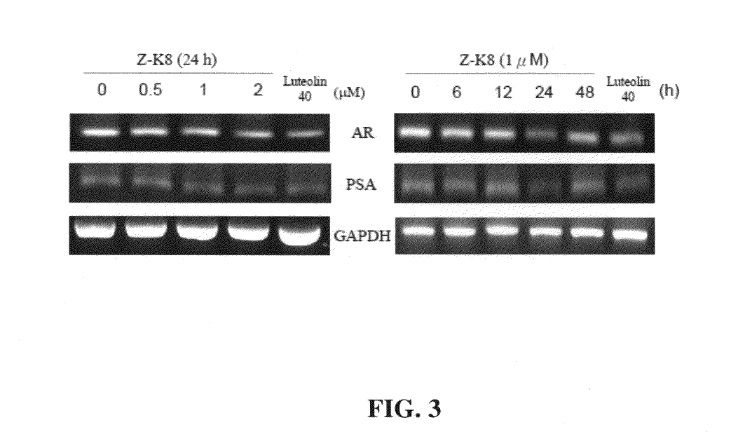

Total RNA from LNCaP, PC3, 3T3 cells was isolated as described above. cDNA was synthesized by reverse transcription of 2 μg of total RNA using oligo(dT)12-18 and SuperScript II RNA reverse transcriptase (Invitrogen). The cDNA was then used as the template to amplify the corresponding DNA fragments by PCR using two sets of synthetic oligonucleotide primers. DNA amplification was performed by PCR with the Thermocycler 2400 (PerkinElmer Life and Analytical Sciences, Boston, Mass.) using the following parameters: 35 cycles of denaturing at 95° C. for 1 min, annealing at 60° C. for 1 min, and extension at 72° C. for 2 min. Primers used for the AR PCR amplifications are (F): 5′-ATG GTG AGC AGA GTG CCC TA-3′ (SEQ ID NO: 1) (R): 5′-GTG GTG CTG GAA GCC TCT CCT-3′ (SEQ ID NO: 2) PSA (F) 5′-GGT GAC CAA GTT CAT GCT GTG-3′ (SEQ ID NO: 3) (R) 5′-GTG TCC TTG ATC CAC TTC CG-3′ (SEQ ID NO: 4) products were separated on 2% agarose gels, stained with ethidium bromide, and visualized using the Fluor Chem imaging system (Alpha InnoTech, San Leandro, Calif.), and levels of glyceraldehyde-3-phosphate dehydrogenase were used as the control. The results are shown in FIGS. 3A and 3B.

5. Cell Cycle Analysis

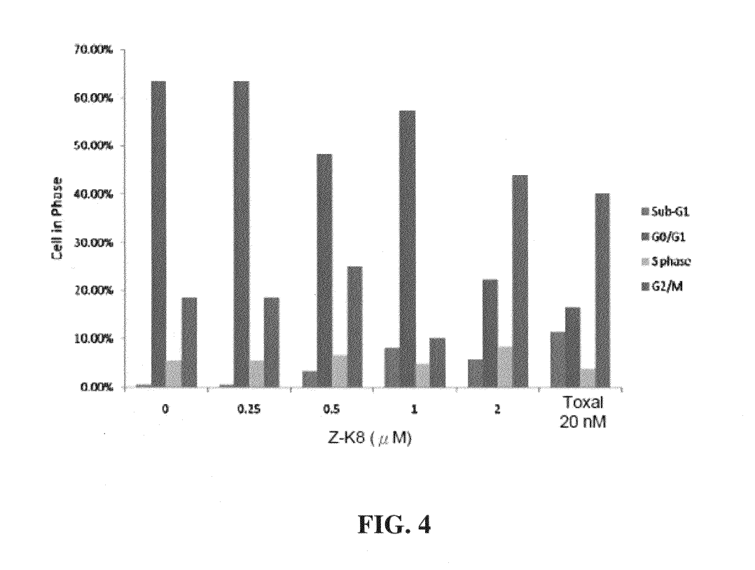

The cell cycle was determined by flow cytometry with DNA staining to reveal the total amount of DNA. Approximately 5×10⁵ of cells were incubated in various concentration of isochaihulactone for the indicated time. Cells were harvested by treating the cells with trypsin/EDTA. The cells were collected, washed with PBS, fixed with cold 70% ethanol overnight, and then stained with a solution containing 45 mg/ml PI, 10 mg/ml RNase A, and 0.1% Triton X-100 for 1 h in the dark. The cells will then pass through FACScan flow cytometer (equipped with a 488-nmargon laser) to measure the DNA content. The data was obtained and analyzed with CellQuest 3.0.1 (Becton Dickinson, Franklin Lakes, N.J.) and ModFitLT V2.0 software. The results are shown Table 4 and FIG. 4.

6. Caspase-3 Activity for Apoptosis Detection

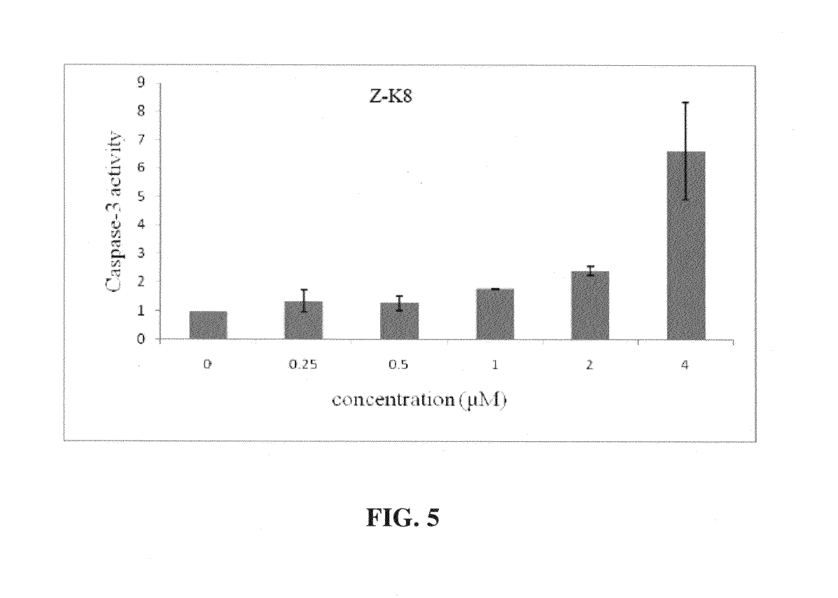

In General, drug can cause tumor apoptosis mediated activate caspase-3 leading to release fluoresce dye to detect by machine. Caspase-3 activity was measured using testis supernatants with the Caspase-Glo Assay System (Promega, Madison, Wis.) according to the manufacturer's instructions. Briefly, 50 μl of the Caspase-Glo buffer was added to 50 μl of tissue supernatants in a 96-well plate and incubated at room temperature for 60 minutes. Luminescence was measured using a Biotek synergy microplate reader (Biotek, Winooski, Vt.). Background activity levels, based on measurements in homogenization buffer only, were measured and subtracted from values in the testis tissue supernatants. All values were normalized to total protein content. Standard curves were used to determine the linearity of the responses. The results are shown in FIG. 5 and Table 5.

7. Antitumor Activity In Vivo

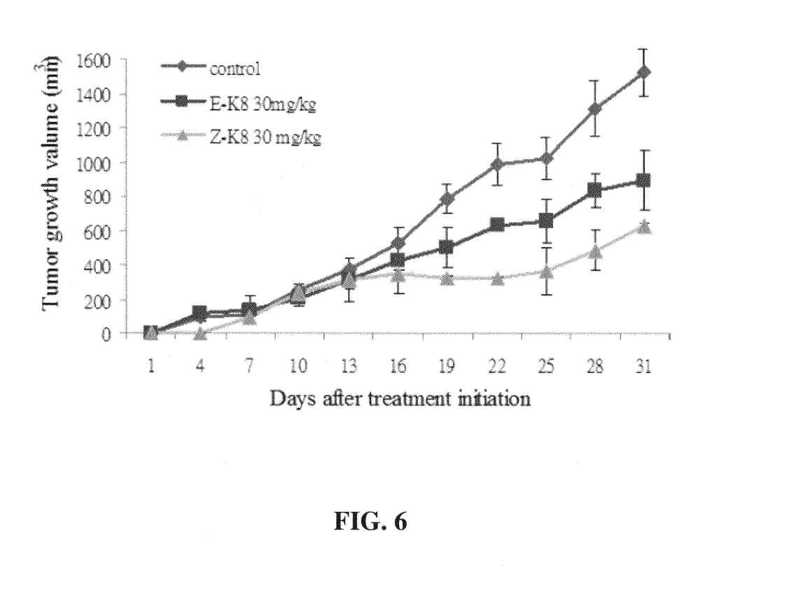

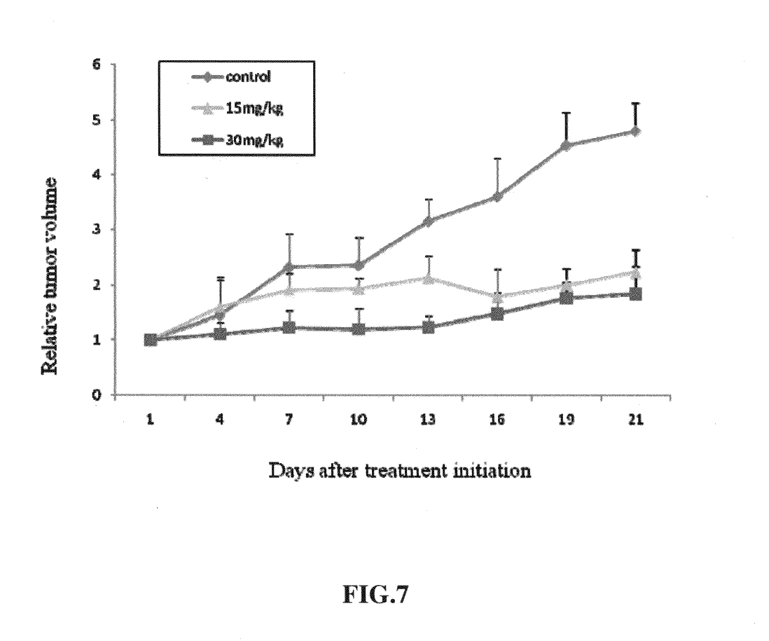

Xenograft mice as a model system to study cytotoxicity effect of isochaihulactone in vivo: the implantation of cancer cells (LNCaP) were carried out similarly to previous reports. Female congenital athymic BALB/c nude (nu/nu) mice were purchased from National Sciences Council (Taipei, Taiwan) and all procedures were performed in compliance with the standard operating procedures of the Laboratory Animal Center of China Medical University (Taichung, Taiwan). All experiments were carried out using 6-8-week old mice weighing 18-22 g. The animals were s.c. implanted with 1×10⁷ cell sinto the back of mice. When the tumor reached 80-120 mm3 in volume, animals were divided randomly into control and test groups consisting of six mice per group (day 0). Daily s.c. administration of z-K8, dissolved in a vehicle of 20% Tween 80 in normal saline (v/v) was performed from days 0 to 4 far from the inoculated tumor sites (>1.5 cm). The control group was treated with vehicle only. The mice were weighed three times a week up to days 21-28 to monitor the effects and the same time the tumor volume was determined by measurement of the length (L) and width (W) of the tumor. The tumor volume at day n (TVn) was calculated as TV (mm3)=(L×W²)/2. The relative tumor volume at day n (RTVn) versus day 0 was expressed according to the following formula: RTVn=TVn/TV0. Tumor regression (T/C(%)) in treated versus control mice was calculated using: T/C(%)=(mean RTV of treated group)/(mean RTV of control group)×100. Xenograft tumors as well as other vital organs of treated and control. The results are shown in Tables 6 and 7 and FIGS. 6 and 7.

8. Immunohistochemical (IHC) Staining

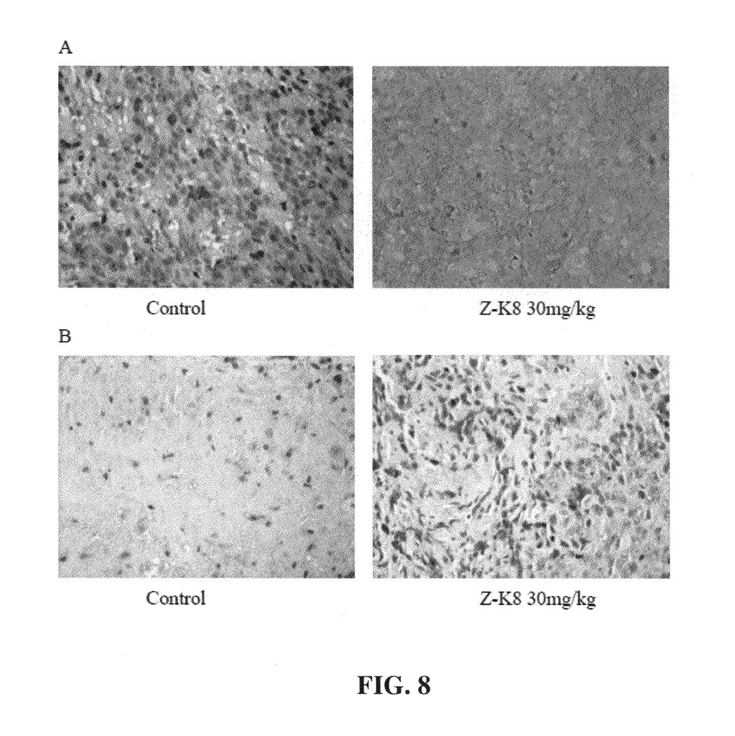

Mice xenograft tissue samples were fixed in 10% formalin and embedded in paraffin. After routine rehydration, antigen retrieval and blocking, the sections were incubated in the primary antibody at 4° C. overnight. The primary antibody was recognized by the biotinylated secondary antibody and visualized by vectastain avidin-biotin complex peroxidase system (ABC kit) as well as peroxidase substrate 3,3′-diaminobenzidine kit (DAB kit). The results are shown in FIGS. 8A and 8B.

9. Statistics

Data were expressed as the mean±SD or SE (standard deviation and standard error, respectively). Statistical significance was analyzed by Student's t-test and Mantel-Cox test. The survival analysis was performed using the Kaplan-Meier method. A P value of <0.05 was considered significant.

### Results

### A. Growth Inhibition Assay

TABLE 1

| | Viability (%) | | |
|---|---|---|---|
| μM | K8 | Z-K8 | E-K8 |
| 0 | 100.0% | 100.0% | 100.0% |
| 2.5 | 67.4% | 33.0% | 86.2% |
| 5 | 43.6% | 18.7% | 72.9% |
| 10 | 40.9% | 17.2% | 69.8% |
| 20 | 21.0% | 16.1% | 65.3% |

TABLE 2

| | |
|---|---|
| Z-K8 | Viability (%) |

| (μM) | LNCaP | PC-3 |
|---|---|---|
| 0 | 100.0% | 100.0% |
| 0.625 | 68.9% | 92.0% |
| 1.25 | 43.7% | 84.5% |
| 2.5 | 21.7% | 74.9% |
| 5 | 18.4% | 44.9% |
| 10 | 16.9% | 28.3% |

Cytotoxicity of K8 (Isochaihulactone) on LNCaP cells was examined in the manner described above. The cells were treated with KS (Isochaihulactone), its Z-form, or its E-form of different concentrations ranging from 2.5 to 20 μM for 24 h. As shown in FIG. 1 and Table 1, both the Z-form and E-form of K8 have anti-tumor effect, but the Z-form of K8 was significantly more effective (IC₅₀=1 μM) than E-K8 on (IC₅₀>20 μM) (P<0.05), and K8, which contain a γ-butyrolactone centred heterocyclic compound forming Z or E configuration at carbon 2.

Cytotoxicities of Z-K8 (Isochaihulactone) on LNCaP (AR-dependent), and PC3 (AR-independent) tumor cell line were examined in the manner described above. The cells were treated with the Z- or E-form K8 (Isochaihulactone) of different concentrations ranging from 0.3125 to 5 μM for 24 h. As shown in FIG. 2 and Table 2, Z-K8 can inhibit the growth of androgen dependent prostate cancer cell line LNCaP as well as that of androgen independent prostate cancer cell line PC3, but the anti-tumor effect of Z-K8 (IC₅₀=1.25 μM) on LNCaP is more significant than that on PC3 (IC₅₀=100 μM). In other words, AR-dependent LNCaP is more sensitive to Z-K8 than AR-independent PC3.

TABLE 3

| | | IC50 (μM) | | |
|---|---|---|---|---|
| cell line | Origin | K8 | E-K8 | Z-K8 |
| NCI-N87 | Gastric carcinoma | ND | ND | >100 |
| HepG2 | Hepatocarcinoma | >20 | ND | ND |
| J5 | Hepatocarcinoma | 21.5 | ND | 2.5 |
| LNCaP | Androgen-dependent, | 4.3 | >20 | 1 |
| | prostate adenocarcinoma | | | |
| PC3 | Androgen-independent, | >100 | >20 | 100 |
| | prostate adenocarcinoma | | | |
| A549 | Lung adenocarcinoma | 18.5 | >100 | 9.8 |
| OECM-1 | Oral squamous cell | ND | >1 | 1 |
| | carcinoma | | | |

As shown in Table 3, both Z-K8 and E-K8 have anti-tumor effects, and the inhibition activity of Z-K8 is higher than E-K8. Thus, these compounds can be used in the treatment of gastric cancer, liver cancer, lung cancer, and oral cancer.

### B. RT-PCR Analysis

RT-PCR was conducted to examine the effect of Z-K8 on several human cancer cells. Briefly, the cells were treated with Z-K8 (0.5 μM to 2 μM) or Luteolin (as a positive control) for 24 h. As shown in FIG. 3A, Z-K8 down regulated AR and PSA in human cancer cells in a dose-dependent manner. In addition, LNCaP cells were treated with 1 μM Z-K8, vehicle (0.2% DMSO), or Luteolin for different times. It was found that Z-K8 decreased AR expression in a time-dependent manner (FIG. 3B). The results suggest that Z-K8 may inhibit the growth of prostate cancer cells by suppressing the expression of AR receptor.

### C. Cell Cycle Analysis

TABLE 4

| | |
|---|---|
| Z-K8 | Cells in phase (%) |

| (μM) | Sub-G1 | G0/G1 | S | G2/M |
|---|---|---|---|---|
| 0 | 0.48% | 63.56% | 5.42% | 18.35% |
| 0.25 | 0.48% | 63.56% | 5.42% | 18.35% |
| 0.5 | 3.24% | 48.30% | 6.61% | 24.99% |
| 1 | 8.19% | 57.27% | 4.70% | 10.16% |
| 2 | 5.73% | 22.25% | 8.39% | 43.92% |
| toxal | 11.43% | 16.40% | 3.95% | 40.23% |
| 20 nM | | | | |

Assays were carried out to examine the effect of Z-K8 in inducing G2/M phase arrest and apoptosis in LNCaP cells. Briefly, the cells were treated with different concentrations of Z-K8 (ranging from 0.25 to 2 μM) for 24 h. The cell cycle analysis was done according to the method described above. As shown in FIG. 4 and Table 4, Z-K8 induced cell cycle G2/M arrest in LNCaP cells. The result shows Z-k8 increases tumor apoptosis (subG1 phase represent cell apoptosis) and G2/M arrest in a dose-dependent manner.

### D. Caspase-3 Activity for Apoptosis Detection

TABLE 5

| Z-K8 | Caspase-3 |
|---|---|
| (μM) | Relative activity |
| 0 | 1 |
| 0.25 | 1.347331119 |
| 0.5 | 1.273829878 |
| 1 | 1.783887813 |
| 2 | 2.398776791 |
| 4 | 6.604113272 |

Assays were carried out to examine whether K8 (Isochaihulactone) increases LNCaP cells caspase-3 activity which leads to tumor cell apoptosis. Briefly, LNCaP cells were treated with different concentrations of Z-K8 (0.25 to 4 μM) for 24 h. The results show that Z-K8 increased caspase-3 activity with dose dependent. As shown in FIG. 5 and Table 5, Z-K8 increases caspase-3 activity in LNCaP cells in a dose-dependent manner, indicating that Z-K8 may promote prostate cancer cell apoptosis by increasing caspase-3 activity. Thus, Z-K8 can be used in the treatment of prostate cancer.

### E. Antitumor Activity In Vivo

TABLE 6

| | Tumor growth volume (mm³) | | | | | | | | | | |
|---|---|---|---|---|---|---|---|---|---|---|---|
| | Days after administration | | | | | | | | | | |
| Group | 1 | 4 | 7 | 10 | 13 | 16 | 19 | 22 | 25 | 28 | 31 |
| control | 0 | 97.5 | 114 | 258 | 378 | 530 | 864 | 977.5 | 1183 | 1334.3 | 1504.3 |
| E-K8 | 0 | 118.0 | 137.1 | 205.7 | 313.7 | 427.4 | 504.7 | 633.5 | 658.6 | 834.9 | 894.9 |
| 30 mg/kg | | | | | | | | | | | |
| Z-K8 | 0 | 0 | 90 | 202.5 | 260 | 255 | 200 | 225 | 375 | 435 | 633.5 |
| 30 mg/kg | | | | | | | | | | | |

TABLE 7

| | Relative tumor volume | | | | | | | |
|---|---|---|---|---|---|---|---|---|
| | Days after administration | | | | | | | |
| Group | 1 | 4 | 7 | 10 | 13 | 16 | 19 | 21 |
| control | 1 | 1.45 | 2.32 | 2.36 | 3.15 | 3.60 | 4.54 | 4.8 |
| Z-K8 | 1 | 1.59 | 1.91 | 1.94 | 2.13 | 1.79 | 2.00 | 2.23 |
| 15 mg/kg | | | | | | | | |
| Z-K8 | 1 | 1.11 | 1.12 | 1.20 | 1.23 | 1.47 | 1.23 | 1.85 |
| 30 mg/kg | | | | | | | | |

Assays were carried out to examine the inhibition of human xenografts growth in vivo by isochaihulactone. Briefly, LNCaP tumor-bearing mice were administered s.c. with vehicle control, 30 mg/kg E-K8, and Z-K8 respectively on days 0-4 for 5 days. The volumes of the tumors were measured. The results are shown in Table 6 and FIG. 6. As shown in FIG. 6, both Z-form and E-form K8 have tumor growth inhibition effect, but Z-form K8 has a better tumor growth inhibition than E-form K8. Scale bars, 100 mm. Data represent means W S.D. of tumor volume at each time

Assays were carried out to examine the inhibition of human xenografts growth in vivo by Z-K8. Again, LNCaP tumor-bearing mice were administered s.c. with vehicle control, Z-K8 15 mg/kg on days 0-4 for 5 days. The volumes of the tumors were measured. The results are shown in FIG. 7 and Table 7. The result shows the tumor volume of therapeutic groups. Scale bars, 100 mm. Data represent means W S.D. of tumor volume at each time point. The result shows that 30 mg/Kg of Z-K8 had a better tumor growth inhibition than 15 mg/kg of Z-K8.

### F. Immunohistochemical (IHC) Staining

Immunohistochemical staining was conducted using a cleaved caspase-3-specific antibody. The results are shown in FIG. 8A. As shown in the figure, the expression of cleaved caspase-3 increased more in the tumors of mice treated with 30 mg/kg Z-K8 than that in the control tumor sections. Similar staining was carried our using an AR-specific antibody. The results (shown in FIG. 8B) revealed that the expression of AR in the tumors of mice treated with 30 mg/kg Z-K8 was weaker than that in the control tumor sections.

The above examples show that Z-K8 and E-K8 may inhibit the growth of androgen dependent or independent prostate cancer cells in vitro or in vivo, and promote the apoptosis of prostate cancer cells. Thus, Z-K8 and E-K8 are useful in the treatment of androgen dependent or independent prostate cancer.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. And, a number of embodiments of the invention have been described. Nevertheless, it will be understood that various modifications may be made without departing from the spirit and scope of the invention. Accordingly, other embodiments are within the scope of the following claims.

What is claimed is:

1. A method of inhibiting the cellular proliferation of at least one selected from the group consisting of androgen dependent prostate cancer cells, androgen independent prostate cancer cells, oral cancer cells, gastric cancer cells, and liver cancer cells (hepatoma) in a subject, comprising administrating to the subject an effective amount of an active component selected from the group consisting of Z form isochaihulactone (Z-K8) of the following formula (I), E form isochaihulactone (E-K8) of the following formula (II), a pharmaceutically acceptable salt of Z-K8 or E-K8, a pharmaceutically acceptable ester of Z-K8 or E-K8, and combinations thereof: 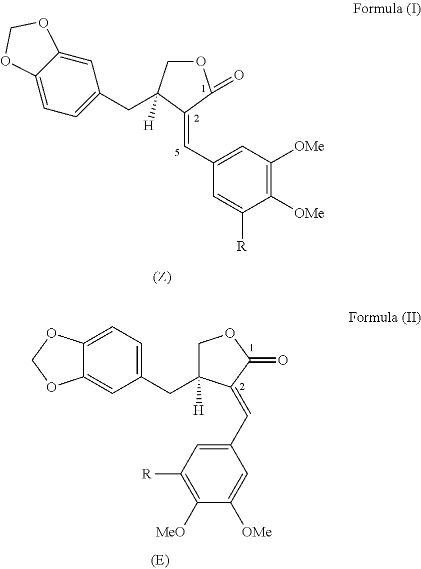
wherein R is H, an alkoxy, or an aryl.

2. The method as claimed in claim 1, wherein R is H, a C₁-C₆ alkoxy, or a C₆-C₂₀ aryl.

3. The method as claimed in claim 2, wherein R is methoxy.

4. The method as claimed in claim 1, wherein the active component is selected from the group consisting of Z-K8, a pharmaceutically acceptable salt of Z-K8, a pharmaceutically acceptable ester of Z-K8, and combinations thereof.

5. The method as claimed in claim 1, which is for inhibiting the cellular proliferation of androgen dependent prostate cancer cells.

6. The method as claimed in claim 1, which is for at least one selected from the group consisting of inhibiting the expression of androgen receptor, inhibiting the expression of prostate specific antigen (PSA), promoting the apoptosis of prostate cancer cells, and activating caspase-3.

7. The method as claimed in claim 1, which is for inhibiting at least one selected from the group consisting of prostate cancer, oral cancer, liver cancer (hepatoma), and gastric cancer.

8. The method as claimed in claim 1, wherein the active component is administrated as a medicament.

9. The method as claimed in claim 1, wherein the subject is human.

10. The method as claimed in claim 9, wherein the active component is Z-K8.

11. The method as claimed in claim 10, wherein the dosage of Z-K8 is about 1 mg/kg-body weight to about 400 mg/kg-body weight per day, based on the total weight of Z-K8.

12. The method as claimed in claim 11, wherein the dosage of Z-K8 is about 15 mg/kg-body weight to about 35 mg/kg-body weight per day, based on the total weight of Z-K8.
